(19) United States
(12) Patent Application Publication
(10) Pub. No.: US 2011/0301042 A1
(43) Pub. Date: Dec. 8, 2011

(54) METHODS OF SAMPLE ENCODING FOR MULTIPLEX ANALYSIS OF SAMPLES BY SINGLE MOLECULE SEQUENCING

(75) Inventors: Kathleen Steinmann, Winchester, MA (US); Elaine Joseph, Cambridge, MA (US); Marie Causey, Belmont, MA (US); John F. Thompson, Warwick, RI (US)

(73) Assignee: HELICOS BIOSCIENCES CORPORATION, Cambridge, MA (US)

(21) Appl. No.: 13/127,669

(22) PCT Filed: Nov. 11, 2009
(86) PCT No.: PCT/US09/64001

§ 371 Date: Aug. 19, 2011

(30) Foreign Application Priority Data
Nov. 11, 2008 (US) 61/113312

(51) Int. Cl. C40B 20/04; C12Q 1/68

(52) U.S. Cl. 506/4; 435/6.1

(57) ABSTRACT

The invention generally relates to methods for sequencing a plurality of nucleic acids from different samples. In certain embodiments, methods of the invention provide contacting a nucleic acid duplex including a primer nucleic acid hybridized to a template nucleic acid with a polymerase enzyme in the presence of a first detectably labeled nucleotide under conditions that permit the polymerase to add nucleotides to the primer in a template-dependent manner, in which a unique oligonucleotide sequence is attached to the template nucleic acid so that the template nucleic acid may be differentiated from other template nucleic acid molecules, detecting a signal from the incorporated labeled nucleotide, and sequentially repeating the contacting and detecting steps at least once, wherein sequential detection of incorporated labeled nucleotide determines the sequence of the nucleic acid.

### RELATED APPLICATION

The present application claims the benefit of and priority to U.S. provisional patent application Ser. No. 61/113,312, filed Nov. 11, 2008, the contents of which are incorporated by reference herein in their entirety.

### FIELD OF THE INVENTION

The invention generally relates to methods for sequencing a plurality of nucleic acids from different samples.

### BACKGROUND

Sequencing-by-synthesis involves template-dependent addition of nucleotides to a template/primer duplex. Nucleotide addition is mediated by a polymerase enzyme and added nucleotides may be labeled in order to facilitate their detection. Single molecule sequencing has been used to obtain high-throughput sequence information on individual DNA or RNA. See, Braslaysky, Proc. Natl. Acad. Sci. USA 100: 3960-64 (2003). Recently, all four Watson-Crick nucleotides may be added simultaneously, each with a different detectable label or nucleotides may be added one at a time in a step-and-repeat manner for imaging incorporations.

One issue that presents itself in sequencing-by-synthesis is the difficulty of tracking template nucleic acid molecules from different samples throughout the sequencing process. Without an ability to correlate which template molecules are from which sample, samples must be analyzed separately, i.e., no multiplexing or pooling of samples is possible. Such a limitation increases costs and decreases throughput for sequencing-by-synthesis platforms.

### SUMMARY

The invention generally relates to methods for sequencing a plurality of nucleic acids from different samples at the same time on the same platform. Methods of the invention accomplish sequencing of a plurality of template nucleic acid molecules from different samples at the same time on the same platform by attaching a unique oligonucleotide sequence (i.e., a bar code) to the template nucleic acid molecules from different samples prior to pooling and sequencing of the template molecules. The bar code allows for template nucleic acid sequences from different samples to be differentiated from each other. Once bar coded, template molecules from different samples may be pooled and sequenced at the same time on the same platform. Because the bar code on each template molecule is sequenced as part of the sequencing reaction, the bar code is a component of the sequence data, and thus the sequence data for different samples is always associated with the sample from which it originated. Due to the association in the sequence data, the sequence data from the pooled samples may be separated after sequencing has occurred and correlated back to the sample from which it originated.

An aspect of the invention provides a method for sequencing a nucleic acid including contacting a nucleic acid duplex including a primer nucleic acid hybridized to a template nucleic acid with a polymerase enzyme in the presence of a first detectably labeled nucleotide under conditions that permit the polymerase to add nucleotides to the primer in a template-dependent manner, in which a unique oligonucleotide sequence is attached to the template nucleic acid so that the template nucleic acid may be differentiated from other template nucleic acid molecules, detecting a signal from the incorporated labeled nucleotide, and sequentially repeating the contacting and detecting steps at least once, wherein sequential detection of incorporated labeled nucleotide determines the sequence of the nucleic acid.

Prior to the contacting step, the method may further include ligating the oligonucleotide sequence to the template molecule. Prior to sequencing, the duplexes may optionally be attached, directly or indirectly, to a substrate. In certain embodiments, at least a portion of the duplexes on the substrate are individually optically resolvable.

The oligonucleotide sequence generally includes certain features that make the sequence useful in sequencing reactions. For example, the oligonucleotide sequence may include a primer site, e.g., a primer sequence such as a polyA tail, that hybridizes to a primer sequence. The oligonucleotide also includes the unique set of nucleotides that allow the template molecule to be distinguished from other template molecules, i.e., the bar code portion of the oligonucleotide. The bar code portion may be any length of nucleotides. In particular embodiments, the bar code portion ranges from about 5 nucleotides to about 15 nucleotides. The oligonucleotide sequence may also include at least one non-natural nucleotide, such as a peptide nucleic acid or a locked nucleic acid, to enhance certain properties of the oligonucleotide. In particular embodiments, the non-natural nucleotide is a locked nucleic acid.

In certain embodiments, the oligonucleotide sequence excludes a terminal thymine on both the 5′ and the 3′ end of the oligonucleotide. In other embodiments, the oligonucleotide sequence excludes any homopolymer region.

Methods of the invention involve detecting a signal from the incorporated labeled nucleotide. Exemplary detectable labels include radiolabels, florescent labels, enzymatic labels, etc. In particular embodiments, the detectable label may be an optically detectable label, such as a fluorescent label. Exemplary fluorescent labels include cyanine, rhodamine, fluorescien, coumarin, BODIPY, alexa, or conjugated multi-dyes.

Another aspect of the invention provides a method for sequencing a plurality of nucleic acids from different samples including contacting a plurality of nucleic acid duplexes including a primer nucleic acid hybridized to a template nucleic acid with a polymerase enzyme in the presence of a first detectably labeled nucleotide under conditions that permit the polymerase to add nucleotides to the primer in a template-dependent manner, in which the template nucleic acids are provided from a plurality of samples, and a unique oligonucleotide sequence is attached to the template nucleic acids from each sample so that the template nucleic acids from the different samples may be differentiated from each other, detecting a signal from the incorporated labeled nucleotide, and sequentially repeating the contacting and detecting steps at least once, wherein sequential detection of incorporated labeled nucleotide determines the sequence of the plurality of nucleic acids. The samples may be obtained from different tissues or body fluids in a single subject. Alternatively, the samples may be obtained from different subjects.

Another aspect of the invention provides a method for sequencing a plurality of nucleic acids from different samples including obtaining template nucleic acids from a plurality of samples, ligating the template nucleic acids from each sample with a unique oligonucleotide sequence so that the template nucleic acids from the different samples may be differentiated from each other, pooling the template nucleic acid molecules from the plurality of samples, hybridizing the pooled template nucleic acid molecules with a plurality of primers to form duplexes including a primer nucleic acid hybridized to a template nucleic acid, contacting the plurality of nucleic acid duplexes with a polymerase enzyme in the presence of a first detectably labeled nucleotide under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the plurality of nucleic acids.

### BRIEF DESCRIPTION OF THE DRAWINGS

FIG. 1 is a schematic shows a method for ligating a bar code oligonucleotide sequence to a template nucleic acid molecule.

FIG. 2 is a schematic showing another method for ligating a bar code oligonucleotide sequence to a template nucleic acid molecule.

### DETAILED DESCRIPTION

The invention generally relates to methods for sequencing a plurality of nucleic acids from different samples. Methods of the invention take a plurality of template nucleic acid molecules from different samples and attach a unique oligonucleotide sequence (i.e., a bar code) to the template nucleic acid molecules from different samples prior to pooling and sequencing of the template molecules. The bar code allows for template nucleic acids from different samples to be differentiated from each other throughout the sequencing process.

The oligonucleotide sequence generally includes certain features that make the sequence useful in sequencing reactions. For example the oligonucleotide sequences are designed to have minimal or no homopolymer regions, i.e., 2 or more of the same base in a row such as AA or CCC, within the unique portion of the oligonucleotide sequence. The oligonucleotide sequences are also designed so that they are at least one edit distance away from the base addition order when performing base-by-base sequencing, ensuring that the first and last base do not match the expected bases of the sequence.

The bar code oligonucleotide sequences may also include blockers, e.g. chain terminating nucleotides, to block base addition to the 3′-end of the template nucleic acid molecules. The oligonucleotide sequences are also designed to have minimal similarity to the base addition order, e.g., if performing a base-by-base sequencing method generally bases are added in the following order one at a time: C, T, A, and G. The oligonucleotide sequence may also include at least one non-natural nucleotide, such as a peptide nucleic acid or a locked nucleic acid, to enhance certain properties of the oligonucleotide.

Depending upon the number of samples to be multiplexed, the bar code portion (unique portion) of the oligonucleotide sequence may be of different lengths. Methods of designing sets of unique oligonucleotide sequences is shown for example in Brenner et al. (U.S. Pat. No. 6,235,475), the contents of which are incorporated by reference herein in their entirety. In certain embodiments, the unique portion of the oligonucleotide sequence ranges from about 5 nucleotides to about 15 nucleotides. In a particular embodiment, the unique portion of the oligonucleotide sequence ranges from about 4 nucleotides to about 7 nucleotides. Since the bar coded portion of the oligonucleotide is sequenced along with the template nucleic acid molecule, the oligonucleotide length should be of minimal length so as to permit the longest read from the template nucleic acid attached. Generally, the unique portion of the oligonucleotide sequence is spaced from the template nucleic acid molecule by at least one base (minimizes homopolymeric combinations).

The oligonucleotide sequence also includes a portion that is used as a primer binding site. The primer binding site may be used to hybridize the now bar coded template nucleic acid molecule to a sequencing primer, which may optionally be anchored to a substrate. The primer binding sequence may be a unique sequence including at least 2 bases but likely contains a unique order of all 4 bases and is generally 20-50 bases in length. One example of a specific sequence binding primer is: 5′-CAG GGC AGA GGA TGG ATG CAA GGA TAA GTG GA-3′ (SEQ ID NO: 1). In a particular embodiment, the primer binding sequence is a homopolymer of a single base, e.g. polyA, generally 20-70 bases in length.

The oligonucleotide sequence also may include a blocker, e.g., a chain terminating nucleotide, on the 3′-end. The blocker prevents unintended sequence information from being obtained using the 3′-end of the primer binding site inadvertently as a second sequencing primer, particularly when using homopolymeric primer sequences. The blocker may be any moiety that prevents a polymerase from adding bases during incubation with a dNTPs. An exemplary blocker is a nucleotide terminator that lacks a 3′-OH, i.e., a dideoxynucleotide (ddNTP). Common nucleotide terminators are 2′,3′-dideoxynucleotides, 3′-aminonucleotides, 3′-deoxynucleotides, 3′-azidonucleotides, acyclonucleotides, etc. The blocker may have attached a detectable label, e.g. a fluorophore. The label may be attached via a labile linkage, e.g., a disulfide, so that following hybridization of the bar coded template nucleic acid to the surface, the locations of the template nucleic acids may be identified by imaging. Generally, the detectable label is removed before commencing with sequencing. Depending upon the linkage, the cleaved product may or may not require further chemical modification to prevent undesirable side reactions, for example following cleavage of a disulfide by TCEP the produced reactive thiol is blocked with iodoacetamide.

Methods of the invention involve attaching the unique oligonucleotide sequences to the template nucleic acid molecules. Template nucleic acids are able to be fragmented or sheared to desired length, e.g. generally from 100 to 500 bases or longer, using a variety of mechanical, chemical and/or enzymatic methods. DNA may be randomly sheared via sonication, e.g. Covaris method, brief exposure to a DNase, or using a mixture of one or more restriction enzymes. RNA may be fragmented by brief exposure to an RNase, heat plus magnesium, or by shearing. The RNA may be converted to cDNA before or after fragmentation.

In certain embodiments, the unique oligonucleotide is attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase (such ligases are available commercially, from New England Biolabs. In a particular embodiment. Methods for using ligases are well known in the art. The polymerase may be any enzyme capable of adding nucleotides to the 3′ terminus of template nucleic acid molecules. The polymerase may be, for example, yeast poly(A) polymerase, commercially available from USB. The polymerase is used according to the manufacturer's instructions.

The ligation may be blunt ended or via use of complementary over hanging ends. In certain embodiments, following fragmentation, the ends of the fragments may be repaired, trimmed (e.g. using an exonuclease), or filled (e.g., using a polymerase and dNTPs), to form blunt ends. Upon generating blunt ends, the ends may be treated with a polymerase and dATP to form a template independent addition to the 3′-end of the fragments, thus producing a single A overhanging. This single A is used to guide ligation of fragments with a single T overhanging from the 5′-end in a method referred to as T-A cloning.

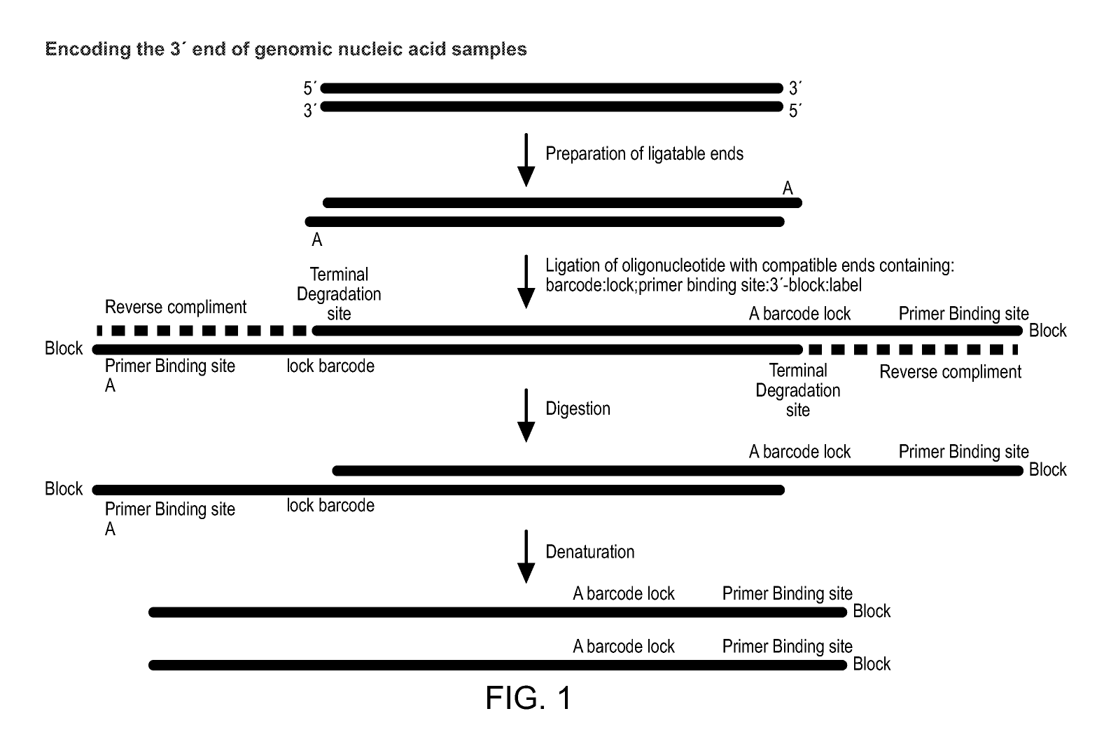

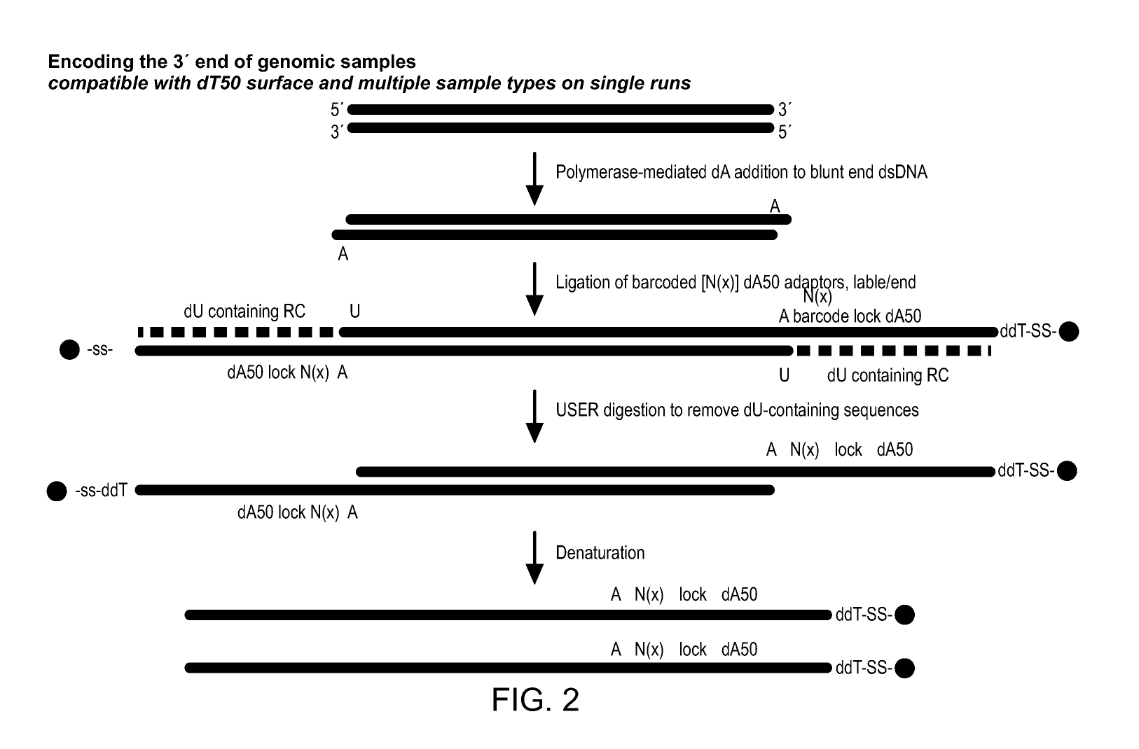

Alternatively, because the possible combination of overhangs left by the restriction enzymes are known after a restriction digestion, the ends may be left as is, i.e., ragged ends. In certain embodiments double stranded oligonucleotides with complementary over hanging ends are used. In a particular example, the A:T single base over hang method is used (see FIGS. 1-2).

In a particular embodiment, the substrate has anchored a reverse complement to the primer binding sequence of the oligonucleotide, for example 5′-TC CAC TTA TCC TTG CAT CCA TCC TCT GCC CTG or a polyT(50). When homopolymeric sequences are used for the primer, it may be advantageous to perform a procedure known in the art as a “fill and lock”. When polyA (20-70) on the sample and polyT (50) on the surface hybridize there is a high likelihood that there will not be perfect alignment, so the hybrid is filled in by incubating the sample with polymerase and TTP. Following the fill step, the sample is washed and the polymerase is incubated with one or two dNTPs complementary to the base(s) used in the lock sequence. The fill and lock can also be performed in a single step process in which polymerase, TTP and one or two reversible terminators (complements of the lock bases) are mixed together and incubated. The reversible terminators stop addition during this stage and can be made functional again (reversal of inhibitory mechanism) by treatments specific to the analogs used. Some reversible terminators have functional blocks on the 3′-OH which need to be removed while others, for example Helicos BioSciences Virtual Terminators have inhibitors attached to the base via a disulfide which can be removed by treatment with TCEP.

In another embodiment, following ligation of the bar code oligonucleotide to the template nucleic acid, the sample is denatured, for example by heating to 95° C. for 5-10 minutes followed by snap cooling to 4° C., and then applied to a surface for hybridization capture. The double stranded barcode oligonucleotide includes features that allow specific degradation of portions of one strand of the hybrid. Desirable locations for such modifications are at base locations closest to the 5′-end of the sample nucleic acid following ligation. Modifications that may be included are the inclusion of one or more uracil residues replacing T's. One, a few, or all of the T's may be substituted with U's in the complementary strand. When using U substitutions, the strand with the U's may be degraded using the enzyme mixture USER (a mixture of Uracil DNA glycosylase (UDG) and the DNA glycosylase-lyase Endonuclease VIII) which creates strand breaks at every U base.

Alternative modifications that may be included are the inclusion of one or more thiophosphate linkages. When using a thiophosphate, the strand can be degraded using a 5′-nuclease degrading bases up until the thiophosphate linkage is reached.

Bar coding of template molecules is useful for all sequencing reactions employing sequencing-by-synthesis approaches but is particularly useful in sequencing methods utilizing single molecule, sequencing-by-synthesis.

In a particular embodiment, the bar code oligonucleotides are used in a single-molecule sequencing-by-synthesis reaction. Single-molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Briefly, a single-stranded nucleic acid (e.g., DNA or cDNA) is hybridized to oligonucleotides attached to a surface of a flow cell. The oligonucleotides may be covalently attached to the surface or various attachments other than covalent linking as known to those of ordinary skill in the art may be employed. Moreover, the attachment may be indirect, e.g., via the polymerases of the invention directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution. The following sections discuss general considerations for nucleic acid sequencing, for example, template considerations, polymerases useful in sequencing-by-synthesis, choice of surfaces, reaction conditions, signal detection and analysis.

### Nucleic Acid Templates

Nucleic acid templates include deoxyribonucleic acid (DNA) and/or ribonucleic acid (RNA). Nucleic acid templates can be synthetic or derived from naturally occurring sources. In one embodiment, nucleic acid template molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples for use in the present invention include viral particles or preparations. Nucleic acid template molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application Publication Number US2002/0190663 A1, published Oct. 9, 2003. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), .beta.-mercaptoethanol, DTE, GSH, cysteine, cysteamine, tricarboxyethyl phosphine (TCEP), or salts of sulfurous acid.

### Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably include a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

### Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9.degree.Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9.degree.Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

### Surfaces

In a preferred embodiment, nucleic acid template molecules are attached to a substrate (also referred to herein as a surface) and subjected to analysis by single-molecule sequencing as described herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. In certain embodiments, the nucleic acid molecule is attached to the substrate through the polymerase molecule. Alternatively, the attachment can be achieved by anchoring a hydrophobic chain into a lipid monolayer or bilayer. Other methods for known in the art for attaching nucleic acid molecules to substrates also can be used.

### Detection

Any detection method can be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophor identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

### Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors. Any of a variety of other alignment techniques known to those of skill in the relevant art may also be used.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for sequencing a nucleic acid, the method comprising:
contacting a nucleic acid duplex comprising a primer nucleic acid hybridized to a template nucleic acid with a polymerase enzyme in the presence of a first detectably labeled nucleotide under conditions that permit the polymerase to add nucleotides to the primer in a template-dependent manner, wherein a unique oligonucleotide sequence is attached to the template nucleic acid so that the template nucleic acid may be differentiated from other template nucleic acid molecules;
detecting a signal from the incorporated labeled nucleotide; and
sequentially repeating the contacting and detecting steps at least once, wherein sequential detection of incorporated labeled nucleotide determines the sequence of the nucleic acid.

2. The method according to claim 1, wherein prior to the contacting step, the method further comprises ligating the oligonucleotide sequence to the template molecule.

3. The method according to claim 1, wherein the oligonucleotide sequence further comprises a primer sequence.

4. The method according to claim 1, wherein the primer sequence is a polyA tail.

5. The method according to claim 3, wherein the oligonucleotide sequence comprises from about 5 nucleotides to about 15 nucleotides excluding the primer sequence.

6. The method according to claim 1, wherein the oligonucleotide sequence excludes a terminal thymine on both the 5′ and the 3′ end of the oligonucleotide.

7. The method according to claim 1, wherein the oligonucleotide sequence excludes any homopolymer region.

8. The method according to claim 1, wherein the oligonucleotide sequence further comprises at least one locked nucleic acid.

9. The method according to claim 1, wherein the duplex is attached to a substrate.

10. The method according to claim 9, wherein at least a portion of the duplexes are individually optically resolvable.

11. The method according to claim 9, wherein the duplex is directly attached to the substrate.

12. The method according to claim 9, wherein the duplex is indirectly attached to the substrate.

13. The method according to claim 1, wherein the detectable label is an optically detectable label.

14. The method according to claim 13, wherein the optically detectable label is a fluorescent label.

15. A method for sequencing a plurality of nucleic acids from different samples, the method comprising:
contacting a plurality of nucleic acid duplexes comprising a primer nucleic acid hybridized to a template nucleic acid with a polymerase enzyme in the presence of a first detectably labeled nucleotide under conditions that permit the polymerase to add nucleotides to the primer in a template-dependent manner, wherein the template nucleic acids are provided from a plurality of samples, and a unique oligonucleotide sequence is attached to the template nucleic acids from each sample so that the template nucleic acids from the different samples may be differentiated from each other;
detecting a signal from the incorporated labeled nucleotide; and
sequentially repeating the contacting and detecting steps at least once, wherein sequential detection of incorporated labeled nucleotide determines the sequence of the plurality of nucleic acids.

16. The method according to claim 15, wherein the samples are obtained from different tissues or body fluids in a single subject.

17. The method according to claim 15, wherein the samples are obtained from different subjects.

18. The method according to claim 15, wherein the duplexes are attached to a substrate.

19. The method according to claim 18, wherein at least a portion of the duplexes are individually optically resolvable.

20. A method for sequencing a plurality of nucleic acids from different samples, the method comprising:
obtaining template nucleic acids from a plurality of samples;
ligating the template nucleic acids from each sample with a unique oligonucleotide sequence so that the template nucleic acids from the different samples may be differentiated from each other;
pooling the template nucleic acid molecules from the plurality of samples;
hybridizing the pooled template nucleic acid molecules with a plurality of primers to form duplexes comprising a primer nucleic acid hybridized to a template nucleic acid;
contacting the plurality of nucleic acid duplexes with a polymerase enzyme in the presence of a first detectably labeled nucleotide under conditions that permit the polymerase to add nucleotides to the primer in a template-dependent manner;
detecting a signal from the incorporated labeled nucleotide; and
sequentially repeating the contacting and detecting steps at least once, wherein sequential detection of incorporated labeled nucleotide determines the sequence of the plurality of nucleic acids.

21. The method according to claim 20, wherein prior to the contacting step, the method further comprises attaching the duplexes to a substrate.

22. The method according to claim 21, wherein at least a portion of the duplexes are individually optically resolvable.

23. The method according to claim 20, wherein the samples are obtained from different tissues or body fluids in a single subject.

24. The method according to claim 20, wherein the samples are obtained from different subjects.


SEQUENCE LISTING (2 sequences)

SEQ ID NO: 1 - DNA, 32 bp, Artificial Sequence
  Synthetic primer
cagggcagaggatggatgcaaggataagtgga

SEQ ID NO: 2 - DNA, 32 bp, Artificial Sequence
  Synthetic primer
tccacttatccttgcatccatcctctgccctg